(19) United States
(12) Patent Application Publication
(10) Pub. No.: US 2025/0332012 A1
(43) Pub. Date: Oct. 30, 2025

(54) FUNNEL CRIMPER WITH TISSUE COMPRESSOR

(71) Applicant: MEDTRONIC, INC., Minneapolis, MN (US)

(72) Inventors: Raymond G. Ryan, Limerick (IE); Constantin F. Ciobanu, Galway (IE); Niall F. Duffy, Galway (IE); Edmond G. Sheahan, Galway (IE); Matthew Case Ziebol, Blaine, MN (US)

(73) Assignee: MEDTRONIC, INC., Minneapolis, MN (US)

(21) Appl. No.: 18/867,754

(22) PCT Filed: Jun. 1, 2023
(86) PCT No.: PCT/IB2023/055634

§ 371 Date: Nov. 20, 2024

Related U.S. Application Data
(60) Provisional application No. 63/348,514, filed on Jun. 3, 2022.

(51) Int. Cl. A61F 2/95

(52) U.S. Cl. CPC A61F 2/9525

(57) ABSTRACT

A funnel crimper includes a body having a tapered portion, an extended outflow portion, and a tissue compressor. The tissue compressor is configured to apply a radially inward force to tissue of a prosthetic valve of a transcatheter heart valve prosthesis disposed on an inner surface of a frame of the transcatheter heart valve prosthesis as the transcatheter heart valve prosthesis is advanced through a lumen of the funnel crimper. The tissue compressor may be a ring of relatively soft material extending into the lumen of the funnel crimper or pressurized fluid applied radially inward to the lumen of the funnel crimper.

### FIELD OF THE INVENTION

The present invention generally relates to a funnel crimping and loading assembly having a tissue compressor to reduce strut crossing, infolding, and/or damage to the tissue of a transcatheter heart valve prosthesis during the crimping process.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

The present disclosure relates to improvements in radially compressing or crimping a heart valve prosthesis to prevent the tissue of the heart valve prosthesis being pinched between struts of a frame the heart valve prosthesis, thereby reducing the risk of damage to the tissue, strut crossing, and infolding during the crimping process.

### BRIEF SUMMARY OF THE INVENTION

In accordance with a first example hereof, a funnel crimper includes a body having a first end, a second end, and a lumen extending from the first end to the second end. The lumen is defined by an inner surface of the body. The body includes a tapered portion and an extended outflow portion, wherein the lumen in the tapered portion tapers from a first diameter at the first end to a second diameter smaller than the first diameter. The extended outflow portion extends from the tapered portion to the second end of the body. The funnel crimper further includes a tissue compressor coupled to the body such that the tissue compressor extends into the lumen relative to the inner surface of the body. The tissue compressor comprises a relatively soft material softer than a relatively hard material of the body configured to apply a radially inward force to tissue of a prosthetic valve of a transcatheter heart valve prosthesis disposed on an inner surface of a frame of the transcatheter heart valve prosthesis as the transcatheter heart valve prosthesis is advanced through the lumen of the body.

In a second example, in the funnel crimper of the first example, the relatively soft material of the tissue compressor has a Shore A hardness in the range of 20 A-80 A.

In a third example, in the funnel crimper of the second example, the relatively soft material of the tissue compressor is selected from the group consisting of closed cell foam, rubber, modified polyurethane, silicone, ethylene propylene diene monomer rubber (EDPM), nitrile, fluorocarbon-based fluoroelastomers (FKM), polychloroprene (neoprene), and/or combinations thereof.

In a fourth example, in the funnel crimper of any one of the first through third examples, the inner surface of the body at the extended outflow portion further comprises a recess, and wherein the tissue compressor is disposed in the recess.

In a fifth example, in the funnel crimper of any one of the first through fourth examples, the tissue compressor is a ring having an outer surface, an inner surface, a thickness defined between the inner surface and the outer surface, a width, and a ring lumen defined by the inner surface of the ring.

In a sixth example, in the funnel crimper of the fifth example, the thickness of the ring is greater than a depth of the recess.

In a seventh example, in the funnel crimper of any one of the first through sixth examples, the tissue compressor further includes a plurality of fingers extending radially inward into the lumen of the body.

In an eighth example, a funnel crimper includes a body having a first end, a second end, and a lumen extending from the first end to the second end. The lumen is defined by an inner surface of the body, the body including a tapered portion and an extended outflow portion, wherein the lumen in the tapered portion tapers from a first diameter at the first end to a second diameter smaller than the first diameter. The extended outflow portion extends form the tapered portion to the second end of the body. The funnel crimper further comprises a tissue compressor configured to apply a radially inward force to tissue of a prosthetic valve of a transcatheter heart valve prosthesis disposed on an inner surface of a frame of the transcatheter heart valve prosthesis as the transcatheter heart valve prosthesis is advanced through the lumen of the body. The tissue compressor comprises a plurality of ports through the extended outflow portion of the body configured to provide pressurized fluid to the lumen of the body.

In a ninth example, in the funnel crimper of the eighth example, the tissue compressor further comprises a ring disposed around an outer surface of the extended outflow portion, the ring defining a channel between an outer surface of the extended outflow portion and an inner surface of the ring, wherein the plurality of ports are disposed within the ring.

In a tenth example, in the funnel crimper of the ninth example, the funnel crimper further comprises an inlet port coupled to the ring, wherein the inlet port is in fluid communication with the channel of the ring.

In an eleventh example, a method of radially compressing a transcatheter heart valve prosthesis comprises the steps of advancing a transcatheter heart valve prosthesis through a tapered portion of a funnel crimper, the tapered portion having a lumen tapering from a first diameter to a second diameter smaller than the first diameter, and the transcatheter heart valve prosthesis including a frame and a prosthetic valve disposed within the frame, advancing the transcatheter heart valve prosthesis through an extended outflow portion of the funnel crimper, the extended outflow portion extending from the tapered portion at the second diameter of the lumen, and applying a radially inward force to the tissue of the prosthetic valve as the transcatheter heart valve prosthesis is advanced through the extended outflow portion such that the tissue is moved radially inward with respect to the frame.

In a twelfth example, in the method of the eleventh example, the method further comprises loading the transcatheter heart valve prosthesis onto or into a delivery catheter system.

In a thirteenth example, in the method of the eleventh example or the twelfth example, the radially inward force is applied through the cells of the frame.

In a fourteenth example, in the method of any one of the eleventh through thirteenth examples, wherein the radially inward force is applied by a relatively soft material extending into the lumen of the funnel crimper relative to an inner surface of the extended outflow portion, and wherein the relatively soft material is softer than a relatively hard material of the funnel crimper.

In a fifteenth example, in the method of the fourteenth example, the relatively soft material has a Shore A hardness in the range of 20 A-80 A.

In a sixteenth example, in the method of the fifteenth example, the relatively soft material is selected from the group consisting of closed cell foam, rubber, modified polyurethane, silicone, ethylene propylene diene monomer rubber (EDPM), nitrile, fluorocarbon-based fluoroelastomers (FKM), polychloroprene (neoprene), and/or combinations thereof.

In a seventeenth example, in the method of any one of the eleventh through thirteenth examples, applying a radially inward force on tissue of the prosthetic valve comprise applying pressurized fluid to the tissue in the extended outflow portion of the funnel crimper.

In an eighteenth example, in the method of the seventeenth example, applying pressurized fluid comprises providing pressurized fluid through ports in the extended outflow portion into the lumen of the funnel crimper.

In a nineteenth example, in the method of the eighteenth example, the pressurized fluid is provided through a ring disposed around the extended outflow portion, the ring defining a channel between an inner surface of the ring and an outer surface of the extended outflow portion, the channel being in fluid communication with the ports.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a side view of an exemplary transcatheter heart valve prosthesis.

FIG. 1B shows a top view of the transcatheter heart valve prosthesis of FIG. 1A.

FIG. 2A shows a perspective side view of a crimping and loading assembly according to embodiments hereof.

FIG. 2B shows a perspective cross-section of the crimping and loading assembly of FIG. 2A, with a plunger thereof disposed within a funnel crimper thereof.

FIG. 2C shows a cross-section of the crimping and loading assembly of FIGS. 2A and 2B.

FIG. 2D shows a close-up view of a portion of the cross-section of FIG. 2C.

FIG. 2E shows a close-up view of a portion of the funnel crimper with the transcatheter heart valve prosthesis being loaded into a delivery catheter.

FIGS. 3A-3B show embodiments of a tissue compressor of the funnel crimper, according to embodiments hereof.

FIG. 4 is a block diagram of a method of using the crimping and loading assembly of FIGS. 2A-2D and 3A-3B according to embodiments hereof.

FIG. 5A shows a perspective side view of a crimping and loading assembly according to embodiments hereof.

FIG. 5B shows a perspective side view of the crimping and loading assembly of FIG. 5A.

FIG. 5C shows a perspective cross-section of the funnel crimper of FIGS. 5A and 5B.

FIG. 5D shows a cross-section of the funnel crimper of FIGS. 5A-5C.

FIG. 5E shows a close-up view of a portion of the cross-section of FIG. 5D.

FIG. 5F shows a close-up view of a portion of the funnel crimper with the transcatheter heart valve prosthesis being loaded into a delivery catheter.

FIG. 6 shows a schematic illustration of the crimping and loading assembly of FIG. 5A, further including a fluid source, according to embodiments hereof.

FIG. 7 is a block diagram that shows a method of using the funnel crimper and the tissue compressor of FIGS. 5A-5F and FIG. 6 according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number or value.

The terms “proximal” and “distal” herein when used with respect to a delivery device, such as a delivery catheter, are used with reference to the clinician using the devices. Therefore, “proximal” and “proximally” mean in the direction toward the clinician, and “distal” and “distally” mean in the direction away from the clinician. The terms “proximal” and “distal” herein when used with respect to a device to be implanted into a body, such as a transcatheter heart valve prosthesis, are used with respect to the direction of blood flow. Therefore, “proximal” and “proximally” mean in the upstream or inflow direction, and “distal” and “distally” mean in the downstream or outflow direction.

As used herein, the term “generally” and “substantially” mean approximately. When used to describe angles such as “substantially parallel” or “substantially perpendicular” the term “substantially” means within 10 degrees of the angle. When used to describe shapes such as “substantially” or “generally” cylindrical or “substantially” or “generally” tube-shaped or “generally” or “substantially” conical, the terms mean that the shape would appear cylindrical or tube-shaped or conical to a person of ordinary skill in the art viewing the shape with a naked eye.

Embodiments hereof relate to a funnel crimper having a tissue compressor configured for use with a transcatheter heart valve prosthesis when radially compressing the transcatheter valve prosthesis into a crimped configuration for loading into a delivery catheter and delivery within a vasculature. More particularly, the tissue compressor is configured to urge or push valve tissue of the transcatheter heart valve prosthesis radially inwards during the crimping process to avoid protrusion of the valve tissue into the frame of the transcatheter heart valve prosthesis that may cause frame infolding, strut crossing, tissue pinching, and/or tissue damage. The tissue compressor is coupled to the funnel crimper and directly contacts the transcatheter heart valve prosthesis during the crimping process such that the tissue compressor may force or push the tissue of the transcatheter heart valve prosthesis radially inwards in relation to the frame of the transcatheter heart valve prosthesis, thereby reducing the chances of the tissue protruding between struts of the frame of the heart valve prosthesis.

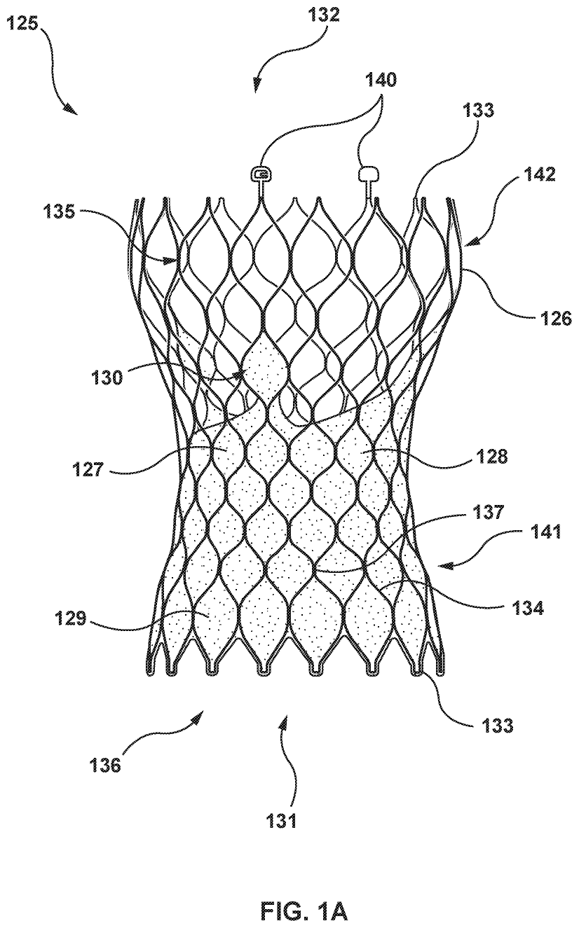

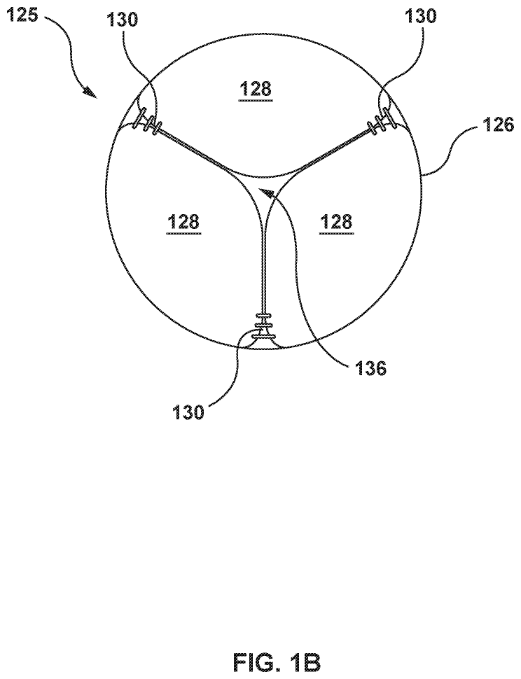

FIGS. 1A and 1B illustrate an example transcatheter heart valve prosthesis 125 that may be utilized with the embodiments of the crimping and loading assembly described herein. The transcatheter heart valve prosthesis 125 is illustrated herein in order to facilitate description of the present invention. The following description of the transcatheter heart valve prosthesis 125 is merely exemplary in nature and is not intended to limit the invention or the application and uses of the invention. It is understood that any number of alternate heart valve prostheses can be used with the crimping and loading assembly and methods described herein. Although the transcatheter heart valve prosthesis 125 is a self-expanding heart valve prosthesis configured for placement within an aortic heart valve, embodiments of the crimping and loading assembly described herein may be utilized with any transcatheter heart valve prosthesis that is crimped onto and/or into a delivery system. For example, embodiments of the crimping and loading assembly described herein may be utilized with a transcatheter heart valve prosthesis configured for placement within a pulmonary, aortic, mitral, or tricuspid valve, or may be utilized with a transcatheter valve prosthesis configured for placement within a venous valve or within other body passageways where it is deemed useful.

FIGS. 1A and 1B illustrate side and top views, respectively, of the transcatheter heart valve prosthesis 125. The transcatheter heart valve prosthesis 125 includes a radially-expandable frame 126 and a prosthetic valve 127. The frame 126 of the transcatheter heart valve prosthesis 125 is a stent or scaffold that supports the prosthetic valve 127 within the interior of the frame 126. The frame 126 may be self-expandable or balloon-expandable. The prosthetic valve 127 includes at least one leaflet 128 disposed within and secured to the frame 126. In an embodiment, the prosthetic valve 127 of the transcatheter heart valve prosthesis 125 includes exactly three leaflets 128, as shown in FIG. 1B. The prosthetic valve 127 of the transcatheter heart valve prosthesis 125 is capable of blocking flow in one direction to regulate flow there-through via the valve leaflets 128. The transcatheter heart valve prosthesis 125 has a radially compressed or crimped configuration for delivery within a vasculature and a radially expanded configuration (as shown in FIGS. 1A and 1B) for deployment within a native heart valve.

As shown in FIG. 1A, the transcatheter heart valve prosthesis 125 includes an inflow portion 141 including an inflow end 131, and an outflow portion 142 including an outflow end 132. The frame 126 of the transcatheter heart valve prosthesis 125 includes a plurality of struts 134 that are arranged to form a plurality of side openings or cells 135 arranged circumferentially around a central longitudinal axis of the transcatheter heart valve prosthesis 125 and longitudinally to form a tubular structure defining a central lumen 136 of the frame 126. Two struts 134 come together to form a crown 133. Four struts 134 come together to form a node 137, as can be seen in FIG. 1A. Each cell 135 of the plurality of cells 135 are defined by four struts 134 and four nodes 137, or three nodes 137 and one crown 133. The frame 126 is configured to secure the prosthetic valve 127 within the central lumen 136 of the frame 126 and to secure the transcatheter heart valve prosthesis 125 in place in the vasculature of the patient.

The inflow end 131 of the frame 126 includes a plurality of crowns 133 with each crown 133 being formed between a pair of adjacent struts 134. Similarly, the outflow end 132 of the frame 126 includes a plurality of crowns 133 with each crown 133 being formed between a pair of adjacent struts 134. The outflow end 132 of the frame 126 may include one or more paddles 140 coupled to one or more crowns 133 at the outflow end 132 of the frame 126 that couple to a delivery system when the transcatheter heart valve prosthesis 125 is to be delivered to a target site. While FIG. 1A illustrates two (2) paddles 140, one skilled in the art will realize that the paddles 140 can be replaced with other components such as eyelets, loops, slots, or any other suitable coupling member, and that more or fewer paddles or other coupling members may be utilized. In some embodiments, the paddles 140 are radiopaque so as to be visible under fluoroscopy, with one of the paddles 140 including a C-shaped marker to assist with orientation of the transcatheter heart valve prosthesis 125 during implantation. Those skilled in the art would recognize that other asymmetric shapes may be utilized so assist in determining the orientation of the transcatheter heart valve prosthesis 125 during implantation. In embodiments, such as the embodiment of FIG. 1A, the paddle 140 with the C-shaped marker is axially aligned with one of the commissures 130 of the valve structure 127, as best seen in FIG. 1A.

In an embodiment, the plurality of cells 135 may be diamond-shaped, as shown in FIG. 1A. In the embodiment described herein, the plurality of cells 135 located at the outflow portion 142 are relatively larger than the plurality of cells 135 located at the inflow portion 141 of the frame 126 to improve access to the coronary arteries. More particularly, the cells 135 located at the outflow portion 142 are configured to be of sufficient size to be easily crossed with a coronary guide catheter into either the right coronary artery or the left main coronary artery once the transcatheter heart valve prosthesis 125 is deployed in situ.

The prosthetic valve 127 of the transcatheter heart valve prosthesis 125 is capable of regulating flow therethrough via the valve leaflets 128 that may form a replacement valve. FIGS. 1A and 1B illustrate an exemplary prosthetic valve 127 having three leaflets 128, although a single leaflet, bicuspid leaflet, or four leaflet configuration may alternatively be used in embodiments hereof. When deployed in situ, the prosthetic valve 127 in a closed state is configured to block blood flow in one direction to regulate blood flow through the central lumen 136 of the frame 126 of the transcatheter heart valve prosthesis 125. FIG. 1A depicts a side view of the transcatheter heart valve prosthesis 125, wherein the prosthetic valve 127 is shown disposed within and secured to the frame 126 of the heart valve prosthesis 125. FIG. 1B depicts an inflow end view of the transcatheter heart valve prosthesis 125 shown in FIG. 1A.

The leaflets 128 may be attached to a graft material or skirt 129 which encloses or lines a portion of the frame 126 as would be known to one of ordinary skill in the art of prosthetic tissue valve construction, for example, using sutures or a suitable biocompatible adhesive. The leaflets 128 are sutured or otherwise securely and sealingly attached along their bases to the interior surface of the graft material, or otherwise attached to the frame 126. Adjoining pairs of leaflets are attached to one another at their lateral ends to form commissures 130, with free edges of the leaflets 128 forming coaptation edges that meet in a closed configuration. The orientation of the leaflets 128 within the prosthetic valve 127 depends on which end of the transcatheter heart valve prosthesis 125 is the inflow end 131 and which end of the transcatheter heart valve prosthesis 125 is the outflow end 132, thereby ensuring one-way flow of blood through the transcatheter heart valve prosthesis 125.

The valve leaflets 128 and graft material may be formed of various flexible materials including, but not limited to, natural material such as tissue from bovine, equine, or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

As shown in FIGS. 1A and 1B, the prosthetic valve 127 includes at least one leaflet 128 disposed within and secured to the frame 126 of the transcatheter heart valve prosthesis 125. A funnel crimper may be used to radially compress the transcatheter heart valve prosthesis 125 from an expanded configuration to a crimped configuration for delivery within a vasculature. During the crimping process, radial compression exerted by the funnel crimper causes the area of the cells 135 of the frame 126 to decrease. In some instances, the tissue of the prosthetic valve 127 may protrude through the cells 135 of the frame 126 during the process, which may cause the tissue to get pinched between the struts 134 of the frame 126 and sustain damage. For example, tissue protrusion may become increasingly likely as the area of the cells 135 increase and/or in view of higher crimping forces that may be needed to crimp balloon expandable implants.

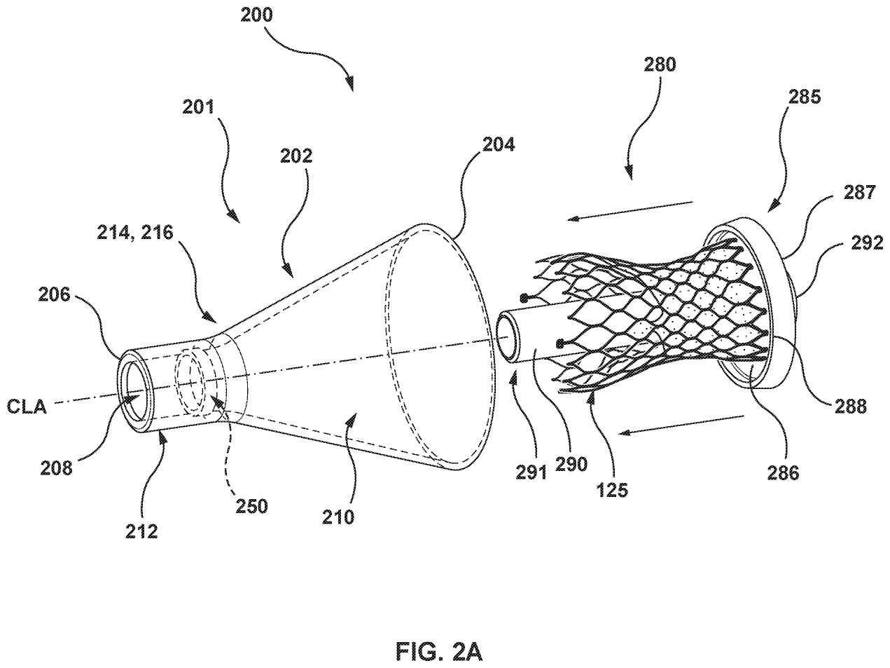

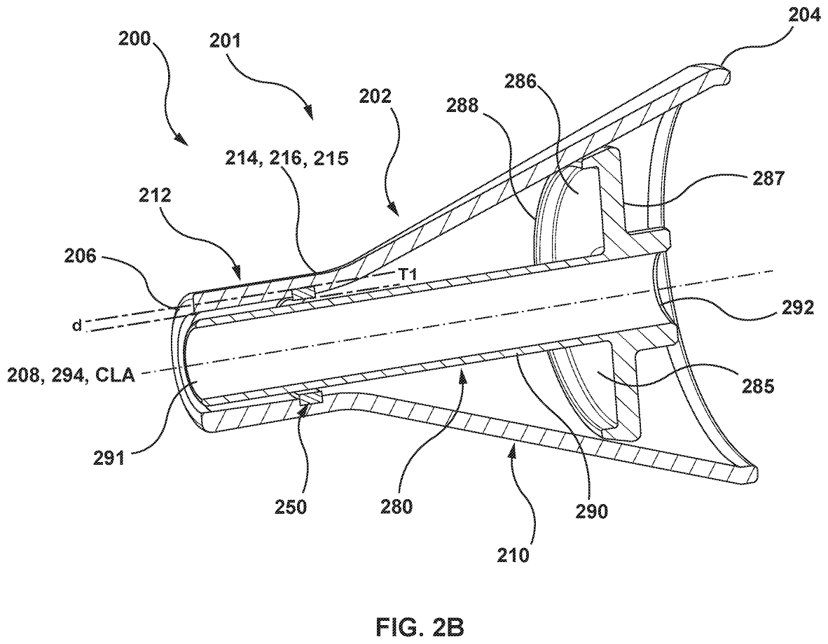

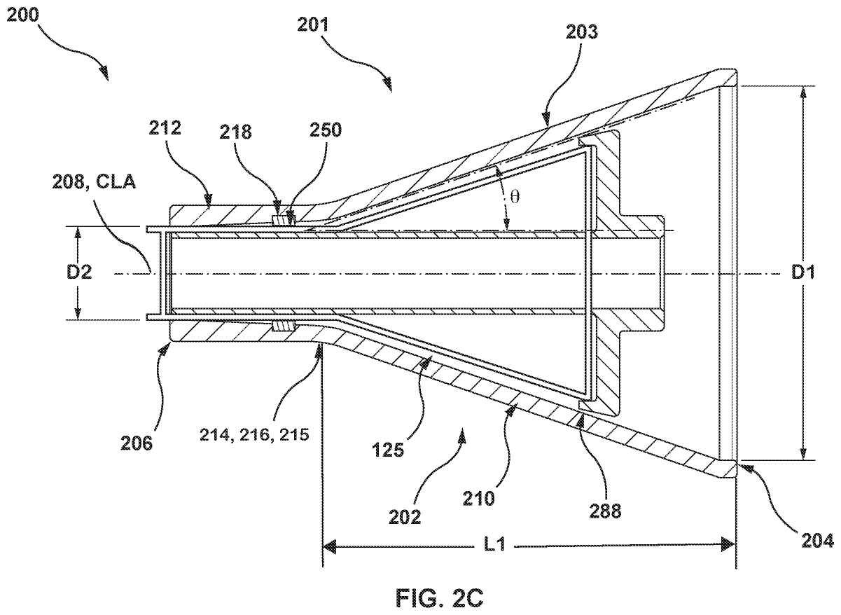

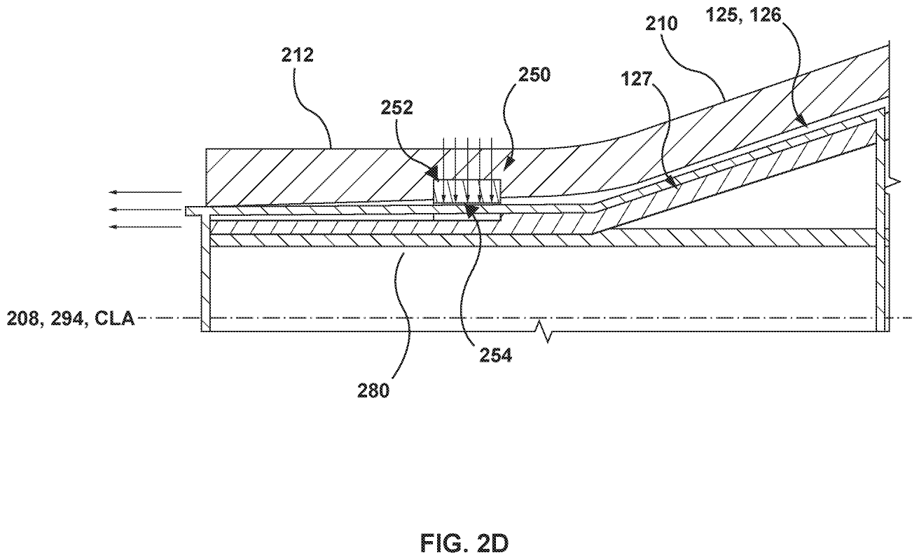

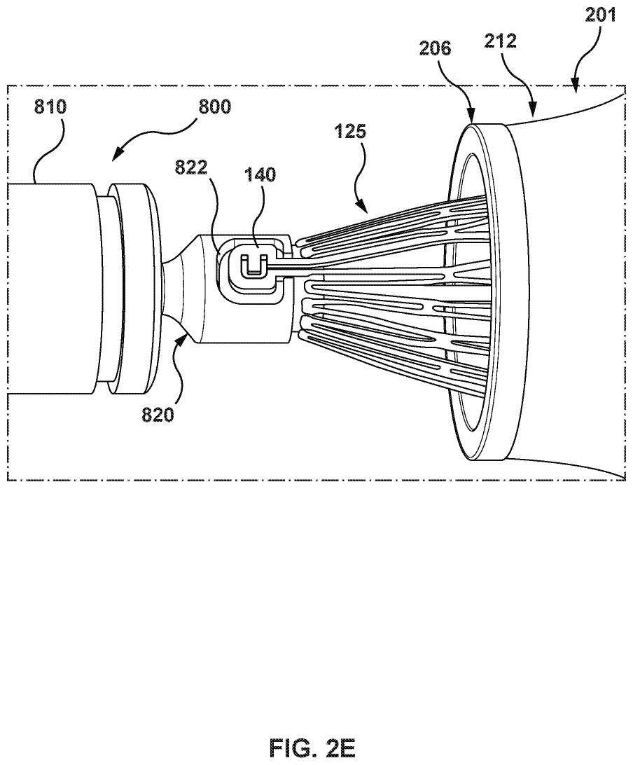

FIGS. 2A-2D are illustrations of a crimping and loading assembly 200 including a funnel crimper 201 and a guide plunger 280. Those skilled in the art would recognize that the crimping and loading assembly 200 may include other parts and/or components not described herein for crimping and loading a transcatheter heart valve prosthesis into a delivery catheter. The crimping and loading assembly 200 is configured for use with a transcatheter valve prosthesis, such as but not limited to the transcatheter heart valve prosthesis 125 described herein, to radially compress the transcatheter valve prosthesis into a crimped configuration and load the transcatheter heart valve prosthesis onto or into a delivery catheter for delivery within a vasculature. For illustrative purposes only, the crimping and loading assembly 200 will be described for use with the transcatheter heart valve prosthesis 125 with the understanding that the crimping and loading assembly 200 may be used with other prosthesis including prosthesis not explicitly disclosed herein.

FIG. 2A-2B show the funnel crimper 201 according to embodiments herein. The funnel crimper 201 includes a body 202 having a first end 204, a second end 206, and a lumen 208 extending therethrough from the first end 204 to the second end 206. The body 202 includes a tapered portion 210 and an extended outflow portion 212 with a boundary 215 defined between the tapered portion 210 and the extended outflow portion 212. The tapered portion 210 extends from the first end 204 of the body 202 to the boundary 215, and the extended outflow portion 212 extends from the boundary 215 to the second end 206 of the body 202. In some embodiments, the body 202 may be a singular component (e.g., molded, machined, extruded) such that the tapered portion 210 and the extended outflow portion 212 are integral and the boundary 215 is continuous. In other embodiments, the body 202 may be provided as multiple, separate components such that the boundary 215 is defined at an interface where a second end 214 of the tapered portion 210 is coupled to a first end 216 of the extended outflow portion 212. In some embodiments, the second end 214 of the tapered portion 210 may be removably coupled to the first end 216 of the extended outflow portion 212 with one or more of a threaded, snap, fastener, or press fit connection. Alternatively, the second end 214 of the tapered portion 210 may be permanently coupled to the first end 216 of the extended outflow portion 212 with one or more of a glued or bonded connection.

As shown in FIGS. 2A-2C, the lumen 208 in the tapered portion 210 has a first diameter at the first end 204 of the body 202 and a second diameter at the second end 214 defined at the boundary 215 that is smaller than the first diameter such that the lumen 208 in the tapered portion 210 tapers in the direction towards the boundary 215. The extended outflow portion 212 is substantially cylindrical in shape such that the lumen 208 in the extended outflow portion 212 has a diameter that is substantially constant and is substantially equal to the second diameter at the second end 214 of the tapered portion 210 defined at the boundary 215. In embodiments, the first diameter D1 is about 25-60 mm and the second diameter D2 is about 3-12 mm. In the embodiment shown, the first end 204 and the second end 206 are both substantially circular in shape. In the embodiment shown and used with the transcatheter heart valve prosthesis 125, the second end 206 is considered the outflow end because the outflow end of the transcatheter heart valve prosthesis 125 is disposed at the second end 206 when being coupled to the delivery catheter.

The tapered portion 210 further has a length L1 of about 20-60 mm from the first end 204 of the body 202 to the second end 214 of the tapered portion 210 defined at the boundary 215. Thus, the length L1 and the diameter reduction from the first end 204 of the body 202 to the second end 214 of the tapered portion 210 results in a crimping angle O of about 10-50 degrees, about 15-25 degrees, or about 20 degrees. As used herein, the crimping angle O means the angle between the inner surface of the tapered portion 210 and the inner surface of the extended outflow portion 212, as shown in FIG. 2C. The smaller crimping angles O disclosed above (i.e., about 10-25 degrees) help reduce strut crossing and infolding.

The funnel crimper 201 further includes a tissue compressor 250 coupled thereto. As shown in FIGS. 2A-2D, the tissue compressor 250 is disposed within a cavity or recess 218 in an inner surface of the extended outflow portion 212 of the funnel crimper 201. In an embodiment, the recess 218 extends around the circumference of the inner surface of the extended outflow portion 212. However, in other embodiments, the recess 218 need not extend around the entire circumference of the extended outflow portion 212. The tissue compressor 250 is a ring shape and is disposed within the recess 218 such that an inner surface 252 of the tissue compressor 250 extends into the lumen 208 of the extended outflow portion 212. For example, as shown in FIG. 2B, in an embodiment, the thickness T1 of the tissue compressor 250 from an outer surface 252 thereof to an inner surface 254 thereof is larger than the depth d of the recess 218.

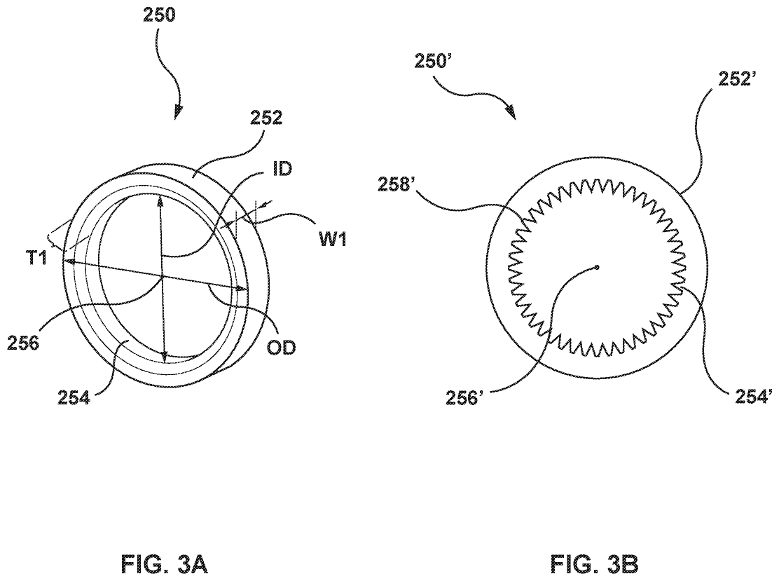

FIGS. 3A-3B show two embodiments of the tissue compressor, labeled as the tissue compressor 250 and the tissue compressor 250′. The tissue compressor 250 shown in FIG. 3A includes the outer surface 252, the inner surface 254, and a central lumen 256 extending therethrough. The tissue compressor 250′ shown in FIG. 3B is similar to the tissue compressor 250 in that it includes an outer surface 252′, an inner surface 254′, and a central lumen 256′. In addition, the tissue compressor 252′ includes fingers 258′ extending inwardly at the inner surface 254′ thereof. As would be understood by those skilled in the art, the tissue compressor 250′ may include more or fewer fingers 258′ than is shown in FIG. 3B. Further, the fingers 258′ may be any shape suitable for the purposes described herein, such as, but not limited to, semi-circular, dome-shaped, dot-shaped, triangular, semi-elliptical, and/or cylindrical. The fingers 258′ may have a length of about 0.5-2.0 mm.

As shown in FIGS. 3A-3B, the tissue compressors 250, 250′ have an inner diameter ID, and outer diameter OD, a width W1, and a thickness T1. The inner diameter ID is smaller than the diameter D2 of the lumen 208 in the extended outflow portion 212 of the funnel compressor 201. Thus, the inner diameter ID of the tissue compressors, 250, 250′ may be in the range of about 1-10 mm. For example, the inner diameter ID of the tissue compressors 250, 250′ may be in the range of 15% to 60% smaller than the second diameter D2 of the lumen 208 in the extended outflow portion 212. The outer diameter OD and the thickness T1 of the tissue compressors 250, 250′ depend on the inner diameter ID and the depth d of the recess 218 in the extended outflow portion 212. In an embodiment, the outer diameter OD may be in the range of 7-20 mm and the thickness T1 may be in the range of 2-4 mm. The width W1 of the tissue compressors 250, 250′ equates to the length along the extended outflow portion 212 that the tissue compressors 250, 250′ extend. In embodiments, the tissue compressors 250, 250′ may have a width W1 in the range of about 2-5 mm.

Compared to a material from which the body 202 is manufactured, the tissue compressors 250, 250′ may include a relatively soft material, such as but not limited to, closed cell foam, rubber, modified polyurethane, silicone, ethylene propylene diene monomer rubber (EDPM), nitrile, fluorocarbon-based fluoroelastomers (FKM) (e.g. VITON), and/or polychloroprene (neoprene) or any combination thereof. In embodiments, the material used for tissue compressors 250, 250′ may have a Shore A hardness in the range of about 20 A-80 A, or in the range of 20 A-60 A, or in the range of 30 A-50 A. In other embodiments, an outer portion of the tissue compressors 250, 250′ may be a material harder than an inner portion of the tissue compressors 250, 250′ that will be in contact with the transcatheter heart valve prosthesis 125, as explained in more detail below. As described in more detail below, the inner portion of the tissue compressor 250, 250′ contacts the transcatheter heart valve prosthesis 125 as the transcatheter heart valve prosthesis 125 moves through the funnel crimper 201 during the crimping and loading procedure.

In the embodiment shown, there is a single tissue compressor 250, 250′ disposed in the extended outflow portion 212 of the funnel crimper 201. However, this is not meant to be limiting. For example, and not by way of limitation, there may be multiple rows of tissue compressors 250, 250′ disposed in the extended outflow portion 212 and/or the tapered portion of the funnel crimper 201.

In some embodiments, the crimping and loading assembly 200 includes a guide plunger 280. FIGS. 2A-2D show an embodiment of the guide plunger 280. The guide plunger 280 guides the transcatheter heart valve prosthesis 125 through the lumen channel 208 of the funnel crimper 201. Those skilled in the art would recognize that other devices for holding and pushing the transcatheter heart valve prosthesis 125 through the funnel crimper 201 may be utilized, and that the guide plunger 280 is merely one exemplary embodiment. The plunger 280 includes a base 285 and a shaft 290. The base 285 of the plunger 280 is a solid, disk-shaped element that includes a first surface 286 and a second surface 287 that opposes the first surface 286. The base 285 is sized and shaped to allow the inflow end 131 of the transcatheter heart valve prosthesis 125 to rest on the first surface 286 of the base 285 in the expanded configuration. In embodiments, the base 285 may include features to hold the inflow end 131 of the transcatheter heart valve prosthesis 125 when in the radially expanded configuration, such as a bumper or lip 288 extending from the first surface 286. In the embodiment shown, the lip 288 protrudes from an entire outer edge of the first surface 286, as best shown in FIG. 2C. The lip 288 is configured to secure the inflow end 131 of the transcatheter heart valve prosthesis 125 in place when the transcatheter heart valve prosthesis 125 is loaded onto the plunger 280. The lip 288 protrudes about 2-6 mm from the first surface 286 of the base 285 and circumscribes the transcatheter heart valve prosthesis 125. In some embodiments, the lip 288 may entirely or partially circumscribe the transcatheter heart valve prosthesis 125. The base 285 of the plunger 280 has a thickness defined between the first surface 286 and the second surface 287 of about 2-6 mm and a diameter of less than 10 mm.

The shaft 290 of the plunger 280 is a longitudinal, cylindrical element that includes a first end 291 and a second end 292. In the embodiment shown, the shaft 290 of the plunger 280 extends through a center of the base 285 such that the first end 281 and the second end 292 are disposed on opposite sides of the base 285. However, this is not meant to be limiting, and the second end 292 may terminate flush with the second surface 287 of the base 285 or more extend beyond the second surface 287 away from the base 285 to function as a surface for a user to grasp the plunger 280. An outer circumference of the first end 291 of the shaft 290 is sized and shaped to fit within the lumen 208 of the extended outflow end 212 of the funnel crimper 201, with sufficient space between an outer surface of the shaft 290 and an inner surface of the extended outflow portion 212 for the transcatheter heart valve prosthesis 125. When the transcatheter heart valve prosthesis 125 is loaded onto the plunger 280, the first end 291 of the shaft 290 extends through the central lumen 136 of the transcatheter heart valve prosthesis 125 through the inflow end 131 until the inflow end 131 of the transcatheter heart valve prosthesis 125 contacts the first surface 286 of the base 285 of the plunger 280, as shown in FIG. 2A. The shaft 290 of the plunger 280 has a longitudinal length of about 30-100 mm and a diameter of about 2.8-11.8 mm. In the embodiment shown the shaft 290 includes a central passageway 294 extending therethrough. The central passageway 294 may be configured to receive a shaft of a delivery catheter therethrough during the crimping and loading process.

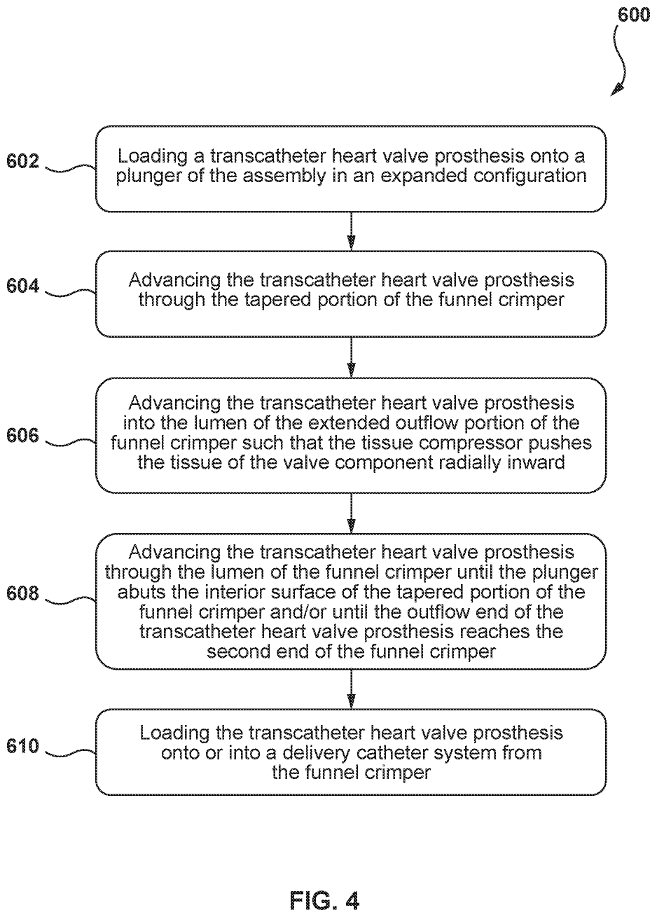

A method of using the crimping loading assembly 200 to crimp the transcatheter heart valve prosthesis 125 and load the transcatheter heart valve prosthesis 125 into and/or onto a delivery catheter will now be described with reference to FIG. 4 and FIGS. 2A-2E. It should be understood that although FIGS. 2A-2D show the tissue compressor 250, the method and FIGS. 2A-2D apply equally to the tissue compressor 250′. For example, the tissue compressor 250′ can replace the tissue compressor 250 in FIGS. 2A-2D. Further, the method is not limited to the transcatheter heart valve prosthesis 125 and can be used with other transcatheter heart valve prostheses. Further, additional or fewer steps of the method described may be used, and some steps may be omitted in this description as being known to those skilled in the art.

Referring to FIG. 4, as shown in step 602 of the method 600, the transcatheter heart valve prosthesis 125 is loaded onto the plunger 280 in an expanded configuration, as shown in FIG. 2A. The first end 291 of the shaft 290 of the plunger 280 is advanced through the central lumen 136 of the transcatheter heart valve prosthesis 125, entering through the inflow end 131, and extends therethrough until the inflow end 131 of the transcatheter heart valve prosthesis 125 rests on the first surface 286 of the base 285, as can be seen in FIG. 2A. In some embodiments, an outer circumference of the inflow end 131 of the transcatheter heart valve prosthesis 125 is displaced radially inward from the circumferential lip 288 of the base 285 such that the transcatheter heart valve prosthesis 125 is secured to the plunger 280 based on the outer circumference of the inflow end 131 exerting an outward radial force against the circumferential lip 288, as best shown in FIG. 2C. Although this step has been described as advancing the shaft 290 of the plunger 280 through the central lumen 136 of the transcatheter heart valve prosthesis 125, the transcatheter heart valve prosthesis 125 could instead be advanced over the shaft 290 towards the base 285, or both the heart valve prosthesis 125 and the plunger 280 may be moved relative to each other to have the inflow end 131 of the transcatheter heart valve prosthesis 125 positioned at the base 285 of the plunger 280.

In a next step 604 of the method 600, the frame 126 of the transcatheter heart valve prosthesis 125 mounted on the plunger 280 is advanced through the lumen 208 of the tapered portion 210 of the funnel crimper 201 to crimp the transcatheter heart valve prosthesis 125, as shown in FIG. 2C.

In a next step 606 of the method 600, the transcatheter heart valve prosthesis 125 continues to be advanced into the lumen 208 of the extended outflow portion 212 such that the tissue compressor 250, 250′ pushes the tissue of the prosthetic valve 127 of the transcatheter heart valve prosthesis 125 radially inward in relation to the frame 126 as the transcatheter heart valve prosthesis 125 passes through the lumen 256, 256′ of the tissue compressor 250, 250′. As the transcatheter heart valve prosthesis 125 is advanced through the lumen 208 of the extended outflow portion 212 and the lumen 250, 250′ of the tissue compressor 250, 250′, the interior surface 254 or the plurality of fingers 258′ of the tissue compressor 250, 250′, respectively, protrude inwardly through the cells 135 of the frame 126 to urge the tissue of the prosthetic valve 127 radially inward, thereby minimizing leaflet protrusion through the cells 135 to avoid pinching or damage thereto and minimize struts crossing and frame infolding, as best shown in FIG. 2D.

In a next step 608 of the method 600, the assembly of the transcatheter heart valve prosthesis 125 and the plunger 280 is advanced through the channel 208 of the funnel crimper 201 along the extended outflow portion 212 towards the second end 206 of the body 202 until the outer surface of the base 285 of the plunger 280 and/or the lip 288 abuts the interior surface of the tapered portion 210 of the funnel crimper 201, as shown in FIGS. 2B and 2C. Once the base 285 of the plunger 280 abuts the interior surface of the tapered portion 210 of the funnel crimper 201, the transcatheter heart valve prosthesis 125 is partially crimped and the paddles 140 on the outflow portion 132 are exposed outside of the second end 206 of the funnel crimper 201 at the end of the extended outflow portion 212. In other embodiments, the base 285 may abut another portion of the funnel crimper 201 or may not abut any portion thereof. In such embodiments, the step 608 is advancing the transcatheter heart valve prosthesis 125 until the outflow end thereof reaches the second end 206 of the funnel crimper 201.

In another step 610 of the method 600, the transcatheter heart valve prosthesis 125 is loaded into a delivery catheter system, for example, a delivery catheter system 800, a portion of which is shown in FIG. 2E. In an exemplary embodiment, the outflow paddles 140 of the frame 126 of the transcatheter heart valve prosthesis 125 are aligned with and inserted into pockets 822 (only one pocket 822 is shown in FIG. 2E) of a spindle or retainer 820 of the delivery catheter system 800. A capsule 810 of the delivery catheter system 800 is advanced over the retainer 820 to retain the outflow paddles 140 in the pockets 822 (not shown). The capsule 820 can abut with or enter the second end 206 of the funnel crimper 201. The transcatheter heart valve prosthesis 125 is then pulled through the funnel crimper 201 and into the capsule by pulling a shaft (not shown) coupled to the retainer 822, thereby crimping the transcatheter heart valve prosthesis 125 as it is pulled through the tapered portion 210 and the extending inflow portion 212, and into the capsule 820 of the delivery catheter system 800 in the compressed configuration. Those skilled in the art would recognize that other delivery catheter systems may be utilized instead of the delivery catheter system 800, and that other methods of loading the transcatheter heart valve prosthesis from the funnel crimper onto or into the delivery catheter system may be utilized. For example, in some embodiments the transcatheter heart valve prosthesis 125 can be pulled through the funnel crimper 201 and/or the funnel crimper 201 can be moved relative to the transcatheter heart valve prosthesis 125 in a direction away from the delivery catheter system 800. In addition, the capsule 810 may be advanced in a direction toward the funnel crimper 201 such that the capsule 125 causes relative movement between the transcatheter heart valve prosthesis 125 and the funnel crimper 201, thereby pushing the funnel crimper 201 away from the retainer 820 of the delivery catheter system 800. Relative motion of the funnel crimper 201 with respect to the transcatheter heart valve prosthesis 125 can thus be achieved while simultaneously covering the prosthesis 125 with the capsule 810.

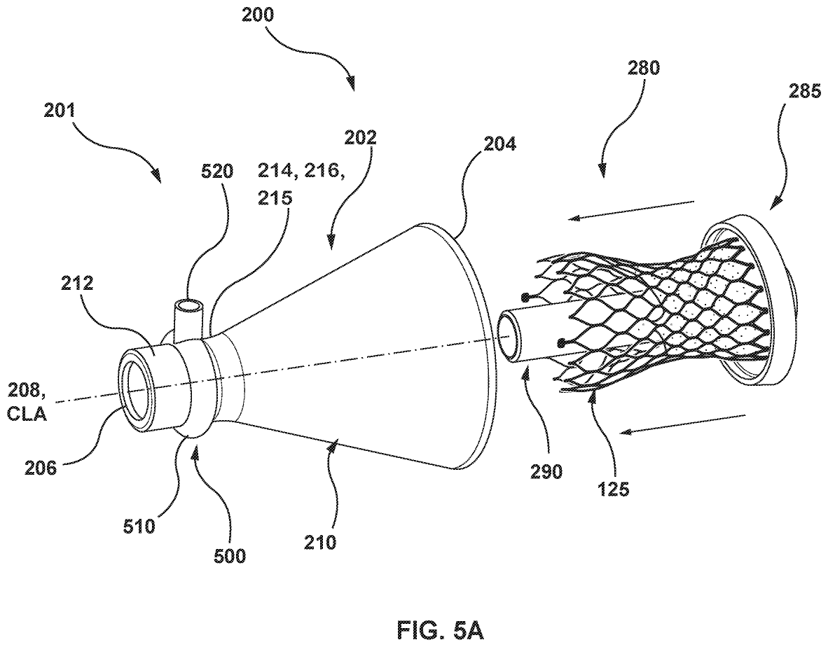

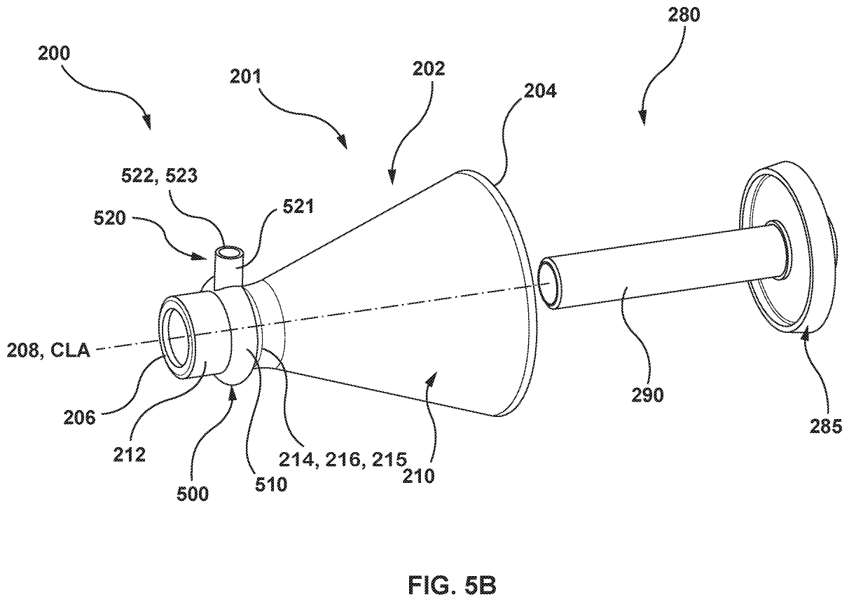

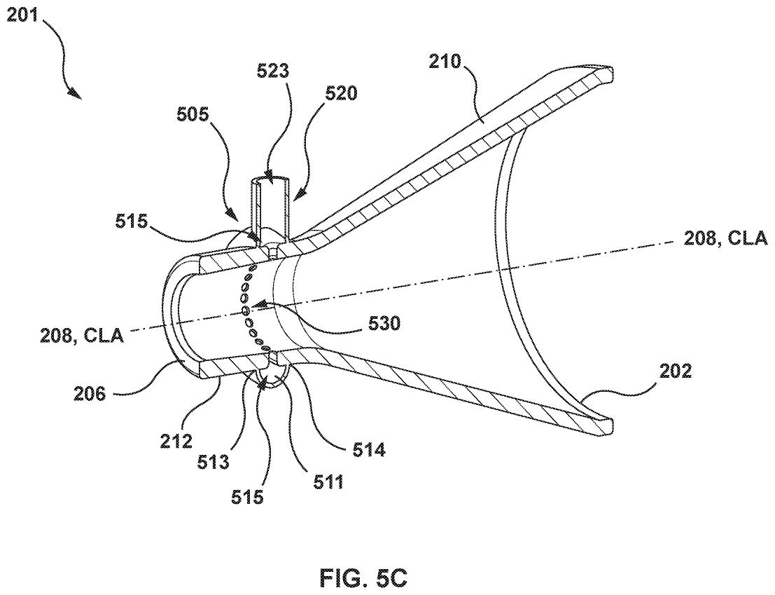

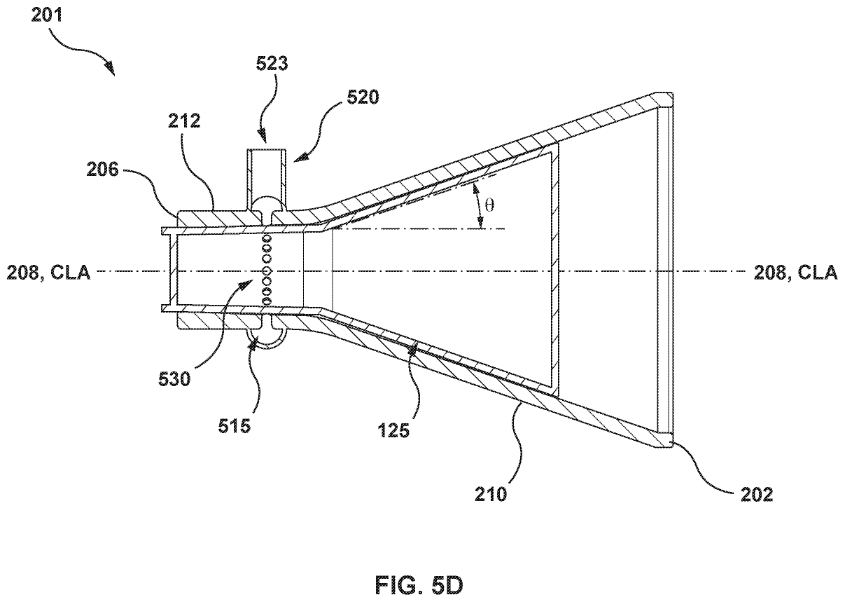

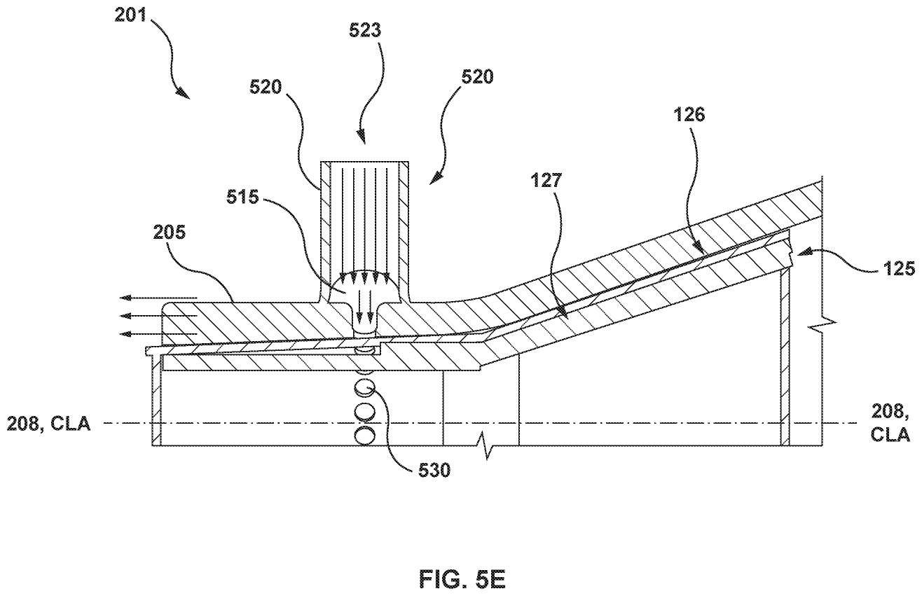

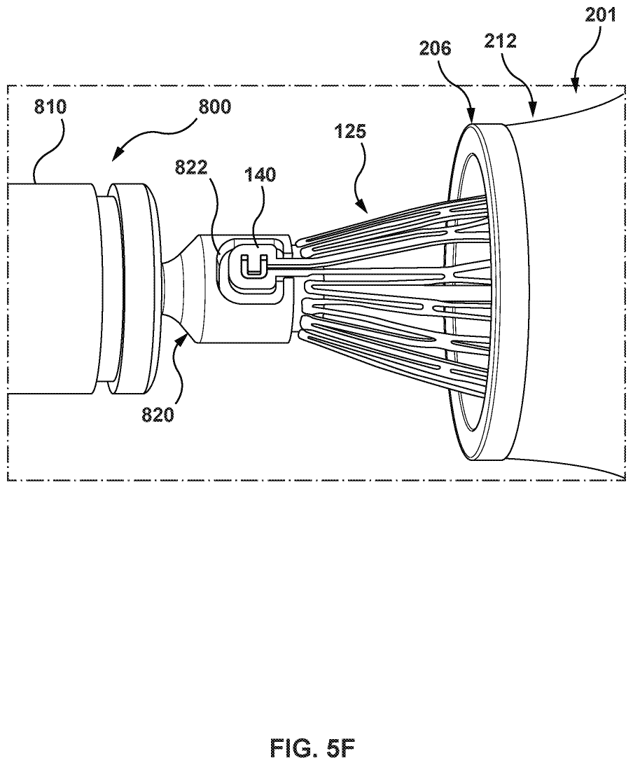

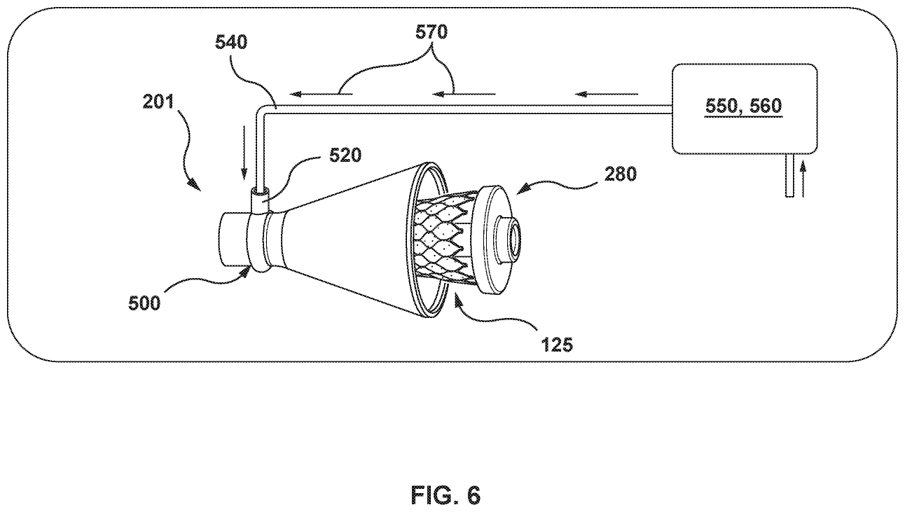

FIGS. 5A-5E and FIG. 6 show the funnel crimper 201 having a having another embodiment of a tissue compressor 500 according to embodiments herein. The funnel crimper 201 of FIGS. 5A-5E and FIG. 6 is the same as the funnel crimper 201 of FIGS. 2A-2E except for the tissue compressor 500 replacing the tissue compressor 250, 250′, and associated changes to the portion of the funnel crimper 201 where the replacement is made. Therefore, details of the funnel crimper 201 that are not changed will not be described again with respect to FIGS. 5A-5E and FIG. 6, and the description above is incorporated into this description of FIGS. 5A-5E and FIG. 6. Further, the plunger 280 will not be described again and the description above is incorporated into this embodiment.

The tissue compressor 500 includes a ring-shaped channel 510, an inlet port 520, and a plurality of circumferentially-spaced perforations our outflow ports 530 in the extended outflow portion 212 of the funnel crimper 201. The tissue compressor 500 is configured to receive fluid 570 and apply the fluid 570 to the tissue of the prosthetic valve 127 of the transcatheter heart valve prosthesis 125 during the crimping process. The term “fluid” as used herein includes gases (i.e., air) as well as liquids such as water, saline, etc. In an embodiment, the fluid 570 is pressurized with a pressure between 5-50 psi.

As noted above, the funnel crimper 201 configured for use with the tissue compressor 500 described herein is similar to the funnel crimper 201 configured for use with the tissue compressor 250, 250′ described above, but instead of having a circumferential cavity or recess 218 in the interior surface of the extended outflow portion 212, the funnel crimper 201 configured for use with the tissue compressor 500 includes a plurality of circumferentially spaced-apart perforations or ports 530 in the extended outflow portion 212 located adjacent to the first end 216 of the extended outflow portion 212, as shown in FIGS. 5C-5E and described in further detail below.

FIG. 5A is an illustration of the crimping and loading assembly 200 including the funnel crimper 201 having the tissue compressor 500 and the guide plunger 280 during the crimping procedure according to embodiments herein. The crimping and loading assembly 200 is configured for use with a transcatheter valve prosthesis, such as but not limited to the transcatheter heart valve prosthesis 125 described herein, when radially compressing the transcatheter heart valve prosthesis 125 into a crimped configuration and loading it into a delivery catheter system for delivery within a vasculature. For illustrative purposes only, the crimping and loading assembly 200 will be described for use with the transcatheter heart valve prosthesis 125 since the structure thereof has been described herein. One skilled in the art would understand that the crimping and loading assembly 200 with the funnel crimper 201 having the tissue compressor 500 can be used with other transcatheter heart valve prostheses.

As can be seen in FIGS. 5A-5C, the tissue compressor 500 includes a ring 505 coupled to an outer surface of the extended outflow portion 212 of the tissue compressor 201. In the embodiment shown, the ring 510 is semi-circular in cross-section such that the circumferential ends 513, 514 of the semi-circle contact the outer surface of the extended outflow portion 212, thereby forming a lumen or channel 515 between an inner surface 511 of the ring 510 and the outer surface of the extended outflow portion 212, as best shown in FIG. 5C. Although the ring 510 is described as being attached to the extended outflow portion 212, this is not meant to be limiting, and the ring 510 may be formed integrally (as one piece) with the extended outflow portion 212. Further, although the ring 510 has been described as being semi-circular in cross-section, that is not mean to be limiting, and any cross-sectional shape that creates the channel 515 may be used.

The tissue compressor 500 further includes an inlet port 520 in fluid communication with the channel 515. The inlet port 520 may be a hollow, cylindrically-shaped tube. However, this is not meant to be limiting, and any inlet port that enables fluid from a fluid source (described in more detail below) to enter the channel 515 of the ring 510 may be utilized. The inlet port 520 defines a lumen 523 in fluid communication with the channel 515.

In an embodiment, the lumen 523 of inlet port 520 may have a diameter defined by an inner surface of the inlet port of about 2-5 mm. In an embodiment, the channel 515 of the ring 510 may have a diameter defined by the outer surface of the extended outflow portion 212 and an inner surface of the ring of about 2-5 mm.

FIG. 5C shows a perspective view of a cross-section of a funnel crimper 201 including the tissue compressor 500 described herein. As mentioned previously above, the funnel crimper 201 includes a plurality of circumferentially-spaced apart ports 530 in the extended outflow portion 212 extending from the interior surface of the extended outflow portion 212 to the exterior surface of the extended outflow portion 212. The ports 530 are aligned with the ring 510 such that the channel 515 of the ring 510 is in fluid communication with the lumen 208 of the extended outflow portion 212 through the ports 530. In the embodiment shown, the ports 530 are substantially circular in shape and extend around the entire circumference of the extended outflow portion 212, but that is not meant to be limiting, and any shape or size of ports suitable for the purposes described herein may be used. In particular, pressurized fluid 570 from a fluid source 550 (see FIG. 6) is delivered to the lumen 523 of the inlet port 520, into the channel 515 of the ring 510, and through the ports 530 into the lumen 508 of the extended outflow portion 212. The pressurized fluid 570 pushes the tissue of the prosthetic valve 127 of the transcatheter heart valve prosthesis 125 radially inward as the prosthesis 125 is advanced through the funnel crimper 201, as described below.

As shown in FIG. 6, the crimping and loading assembly 200 further includes a fluid source 550 for providing fluid 570 to the funnel crimper 201. In the embodiment shown, a pump 560 sends the fluid 570 through tubing 540 to the inlet port 520 described above, to provide pressurized fluid 570 through the ports 530 into the lumen 208 of the extended outflow portion 212 of the funnel crimper 201. One skilled in the art will realize that FIG. 6 illustrates one example of a system to provide pressurized fluid to the funnel crimper 201 and that components illustrated in FIG. 6 may be removed and/or additional components may be added.

In the embodiment shown, there is a single row of circumferentially-spaced apart ports 530 in the extended outflow portion 212 of the funnel crimper 201. However, this is not meant to be limiting. For example, and not by way of limitation, there may be multiple rows of circumferentially-spaced apart ports 530 in the extended outflow portion 212 and/or the tapered portion of the funnel crimper 201.

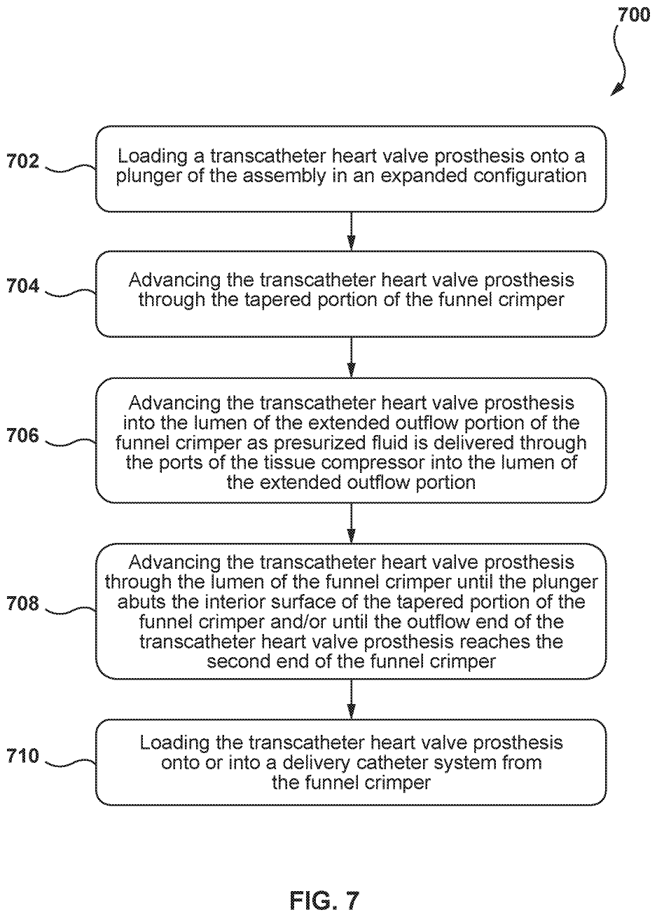

A method of using the crimping loading assembly 200 including the crimper 201 with the tissue compressor 500 to crimp the transcatheter heart valve prosthesis 125 and load the transcatheter heart valve prosthesis 125 into and/or onto a delivery catheter will now be described with reference to FIG. 7, FIGS. 5A-5F, and FIG. 6. It should be understood that the method is not limited to the transcatheter heart valve prosthesis 125 and can be used with other transcatheter heart valve prostheses. Further, additional or fewer steps of the method described may be used, and some steps may be omitted in this description as being known to those skilled in the art.

In this embodiment, as shown in step 702 of the method 700, the transcatheter heart valve prosthesis 125 is loaded onto the plunger 280 in an expanded configuration, as shown in FIG. 2A. The first end 291 of the shaft 290 of the plunger 280 is advanced through the central lumen 136 of the transcatheter heart valve prosthesis 125, entering through the inflow end 131, and extends therethrough until the inflow end 131 of the transcatheter heart valve prosthesis 125 rests on the first surface 286 of the base 285, as can be seen in FIG. 2C (which applies to this embodiment). An outer circumference of the inflow end 131 of the transcatheter heart valve prosthesis 125 is displaced radially inward from the circumferential lip 288 of the base 285 such that the transcatheter heart valve prosthesis 125 is secured to the plunger 280, as best shown in FIG. 2C. Although this step has been described as advancing the shaft 290 of the plunger 280 through the central lumen 136 of the transcatheter heart valve prosthesis 125, the transcatheter heart valve prosthesis 125 could instead be advanced over the shaft 290 towards the base 285, or both the heart valve prosthesis 125 and the plunger 280 may be moved relative to each other to have the inflow end 131 of the transcatheter heart valve prosthesis 125 positioned at the base 285 of the plunger 280.

In a next step 704 of the method 700, the frame 126 of the transcatheter heart valve prosthesis 125 mounted on the plunger 280 is advanced through the lumen 208 of the tapered portion 210 of the funnel crimper 201 to crimp the transcatheter heart valve prosthesis 125, as shown in FIG. 5D (the plunger 280 is not shown in FIG. 5D for clarity).

In a next step 706 of the method 700, the transcatheter heart valve prosthesis 125 continues to be advanced into the lumen 208 of the extended outflow portion 212 of the funnel crimper 201. Prior to or as the transcatheter heart valve prosthesis is being advanced through the extended outflow portion 212, pressurized fluid 570, such as from the fluid source 560 shown in FIG. 6, is provided into the inlet port 520, into the channel 515 of the ring 510, and into the lumen 208 of the extended outflow portion 212 through the ports 530. Thus, the pressurized fluid 570 pushes the tissue of the prosthetic valve 127 of the transcatheter heart valve prosthesis 125 radially inward in relation to the frame 126 as the transcatheter heart valve prosthesis 125 passes through the lumen 280 of the extended outflow portion 212 of the funnel crimper 201, thereby minimizing leaflet protrusion through the cells 135 of the frame 126 to avoid pinching or damage thereto and minimize struts crossing and frame infolding, as best shown in FIG. 5E.

In a next step 708 of the method 700, the assembly of the transcatheter heart valve prosthesis 125 and the plunger 280 is advanced through the channel 208 of the funnel crimper 201 along the extended outflow portion 212 towards the second end 206 of the body 202 until the outer surface of the base 285 of the plunger 280 and/or the lip 288 abuts the interior surface of the tapered portion 210 of the funnel crimper 201, as shown in FIGS. 5D and 5E. Once the base 285 of the plunger 280 abuts the interior surface of the tapered portion 210 of the funnel crimper 201, the transcatheter heart valve prosthesis 125 is partially crimped and the paddles 140 on the outflow portion 132 are exposed outside of the second end 206 of the funnel crimper 201 at the end of the extended outflow portion 212. In other embodiments, the base 285 may abut another portion of the funnel crimper 201 or may not abut any portion thereof. In such embodiments, the step 708 is advancing the transcatheter heart valve prosthesis 125 until the outflow end thereof reaches the second end 206 of the funnel crimper 201. During this continued advancement, the pressurized fluid 570 continues to be push the tissue of the prosthetic valve 125 radially inward, as discussed above.

In another step 710 of the method 700, the transcatheter heart valve prosthesis 125 is loaded into a delivery catheter system, for example, a delivery catheter system 800, a portion of which is shown in FIG. 5F. In an exemplary embodiment, the outflow paddles 140 of the frame 126 of the transcatheter heart valve prosthesis 125 are aligned with and inserted into pockets 822 (only one pocket 822 is shown in FIG. 2E) of a spindle or retainer 820 of the delivery catheter system 800. A capsule 810 of the delivery catheter system 800 is advanced over the retainer 820 to retain the outflow paddles 140 in the pockets 822 (not shown). The capsule 820 can abut with or enter the second end 206 of the funnel crimper 201. The transcatheter heart valve prosthesis 125 is then pulled through the funnel crimper 201 and into the capsule 820 by pulling a shaft (not shown) coupled to the retainer 822, thereby crimping the transcatheter heart valve prosthesis 125 as it is pulled through the tapered portion 210 and the extending inflow portion 212, and into the capsule 820 of the delivery catheter system 800 in the compressed configuration. The pressurized fluid 570 may continue to be provided as the transcatheter heart valve prosthesis 125 is loaded into the delivery catheter system 800, or the pressurized fluid could stop being provided at any time during the loading process if it is no longer needed to push the tissue of the prosthetic valve 127 inwardly. Those skilled in the art would recognize that other delivery catheter systems may be utilized instead of the delivery catheter system 800, and that other methods of loading the transcatheter heart valve prosthesis from the funnel crimper onto or into the delivery catheter system may be utilized.

It should be understood that the crimping and loading assemblies described herein is merely exemplary in nature and is not intended to limit the invention or the application and uses of the invention. It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

1. A funnel crimper comprising:
a body having a first end, a second end, and a lumen extending from the first end to the second end, the lumen being defined by an inner surface of the body, the body including a tapered portion and an extended outflow portion, wherein the lumen in the tapered portion tapers from a first diameter at the first end to a second diameter smaller than the first diameter, and wherein the extended outflow portion extends from the tapered portion to the second end of the body; and
a tissue compressor coupled to the body such that the tissue compressor extends into the lumen relative to the inner surface of the body, wherein the tissue compressor comprises a relatively soft material softer than a relatively hard material of the body, and wherein the tissue compressor is configured to apply a radially inward force to tissue of a prosthetic valve of a transcatheter heart valve prosthesis disposed on an inner surface of a frame of the transcatheter heart valve prosthesis as the transcatheter heart valve prosthesis is advanced through the lumen of the body.

2. The funnel crimper of claim 1, wherein the relatively soft material of the tissue compressor has a Shore A hardness in the range of 20 A-80 A.

3. The funnel crimper of claim 2, wherein the relatively soft material of the tissue compressor is selected from the group consisting of closed cell foam, rubber, modified polyurethane, silicone, ethylene propylene diene monomer rubber (EDPM), nitrile, fluorocarbon-based fluoroelastomers (FKM), polychloroprene (neoprene), and/or combinations thereof.

4. The funnel crimper of claim 1, wherein the inner surface of the body at the extended outflow portion further comprises a recess, and wherein the tissue compressor is disposed in the recess.

5. The funnel crimper of claim 1, wherein the tissue compressor is a ring having an outer surface, an inner surface, a thickness defined between the inner surface and the outer surface, a width, and a ring lumen defined by the inner surface of the ring.

6. The funnel crimper of claim 5, wherein the thickness of the ring is greater than a depth of the recess.

7. The funnel crimper of claim 6, wherein the tissue compressor further includes a plurality of fingers extending radially inward into the lumen of the body.

8. A funnel crimper comprising:
a body having a first end, a second end, and a lumen extending from the first end to the second end, the lumen being defined by an inner surface of the body, the body including a tapered portion and an extended outflow portion, wherein the lumen in the tapered portion tapers from a first diameter at the first end to a second diameter smaller than the first diameter, and wherein the extended outflow portion extends from the tapered portion to the second end of the body; and
a tissue compressor configured to apply a radially inward force to tissue of a prosthetic valve of a transcatheter heart valve prosthesis disposed on an inner surface of a frame of the transcatheter heart valve prosthesis as the transcatheter heart valve prosthesis is advanced through the lumen of the body, the tissue compressor comprising a plurality of ports through the extended outflow portion of the body configured to provide pressurized fluid to the lumen of the body.

9. The funnel crimper of claim 8, wherein the tissue compressor further comprises a ring disposed around an outer surface of the extended outflow portion, the ring defining a channel between an outer surface of the extended outflow portion and an inner surface of the ring, wherein the plurality of ports are disposed within the ring.

10. The funnel crimper of claim 9, further comprising an inlet port coupled to the ring, wherein the inlet port is in fluid communication with the channel of the ring.

11. A method of radially compressing a transcatheter heart valve prosthesis comprising:
advancing a transcatheter heart valve prosthesis through a tapered portion of a funnel crimper, the tapered portion having a lumen tapering from a first diameter to a second diameter smaller than the first diameter, the transcatheter heart valve prosthesis including a frame and a prosthetic valve disposed within the frame;
advancing the transcatheter heart valve prosthesis through an extended outflow portion of the funnel crimper, the extended outflow portion extending from the tapered portion at the second diameter of the lumen; and
applying a radially inward force to tissue of the prosthetic valve as the transcatheter heart valve prosthesis is advanced through the extended outflow portion such that the tissue is moved radially inward with respect to the frame.

12. The method of claim 11, further comprising loading the transcatheter heart valve prosthesis onto or into a delivery catheter system.

13. The method claim 11, wherein the radially inward force is applied through cells of the frame.

14. The method of claim 11, wherein the radially inward force is applied by a relatively soft material extending into the lumen of the funnel crimper relative to an inner surface of the extended outflow portion, and wherein the relatively soft material is softer than a relatively hard material of the funnel crimper.

15. The method of claim 14, wherein the relatively soft material has a Shore A hardness in the range of 20 A-80 A.

16. The method of claim 15, wherein the relatively soft material is selected from the group consisting of closed cell foam, rubber, modified polyurethane, silicone, ethylene propylene diene monomer rubber (EDPM), nitrile, fluorocarbon-based fluoroelastomers (FKM), polychloroprene (neoprene), and/or combinations thereof.

17. The method of claim 11, wherein applying a radially inward force on tissue of the prosthetic valve comprises applying pressurized fluid to the tissue in the extended outflow portion of the funnel crimper.

18. The method of claim 17, wherein applying pressurized fluid comprises providing pressurized fluid through ports in the extended outflow portion into the lumen of the funnel crimper.

19. The method of claim 18, wherein the pressurized fluid is provided through a ring disposed around the extended outflow portion, the ring defining a channel between an inner surface of the ring and an outer surface of the extended outflow portion, the channel being in fluid communication with the ports.
